(19) United States
(12) Patent Application Publication
(10) Pub. No.: US 2026/0276589 A1
(43) Pub. Date: Sep. 17, 2026

(54) Method and System for Detecting and Quantifying Broken Steel Wire Wraps in Prestressed Concrete Cylinder Pipe (PCCP)

(71) Applicant: SafeNet International LLC, Arlington Heights, IL (US)

(72) Inventor: Joseph Y. Fang, South Barrington, IL (US)

(21) Appl. No.: 19/076,657

(22) Filed: Mar. 11, 2025

(51) Int. Cl. G01N 27/82; B25J 5/02; B25J 19/02; G01N 33/2045

(52) U.S. Cl. CPC G01N 27/82; B25J 5/02; B25J 19/027; G01N 33/2045

(57) ABSTRACT

A system and method using a pipe robot in network PCCP to carry electromagnetic detection equipment for inspection and diagnostic routines. The system is able to diagnose PCCP broken steel wire(s) and improve diagnostic speed 10× without losing accuracy. The system can be trained by data to adapt to different PCCP pipeline. The system replaces on-site professionals, greatly improves the detection speed, and is able to identify PCCP broken steel wire(s) in real time. An optimal configuration of the emitter and the receiver coil offset distance, the emitter signal frequency, and the frequency of acquisition is used in order to increase diagnostic speed.

### TECHNICAL FIELD OF THE INVENTION

The present invention relates to both a system and method for rapidly detecting structural defects, such as broken tensioned steel wire wraps in prestressed concrete cylinder pipe (PCCP) in the absence of professional operators. The method and system may also relate to quantifying the number of broken steel wire wraps as well.

### BACKGROUND OF THE INVENTION

Prestressed Concrete Cylinder Pipe, or PCCP is commonly used in large-scale water distribution systems due to its high-pressure resistance, long lifespan, and ability to withstand external loads from soil conditions.

As illustrated in FIG. 1, PCCP is made by creating a concrete core, then wrapping a thin steel cylinder around it, and finally applying high-tensile steel wires in a helical pattern around the concrete core, essentially “pre-stressing” it to withstand high internal pressure; the two ends of the steel cylinder are connected with steel bearing, socket rings, for connection between adjacent PCCP pipes and align the centers of the adjacent pipes; the whole pipe is then coated with a layer of cement mortar to protect the steel components from corrosion. The concrete core provides compressive strength while the steel cylinder acts as a watertight barrier and the prestressing wires provide the necessary tensile strength to resist pressure from within the pipe to prevent bursting.

When pressure builds inside the pipe, the prestressing wires counteract the outward force by applying inward tension, effectively distributing the load across the entire pipe structure. The concrete core resists compression while the steel wires handle tension, maximizing the pipe's ability to withstand pressure. The steel cylinder ensures that water stays contained within the pipe, preventing leaks.

There are different types of PCCP, including “lined cylinder pipe” where the steel cylinder is directly wrapped with prestressing wires (directionally spring tightened), and “embedded cylinder pipe” where the steel cylinder is encased within the concrete core. Proper manufacturing techniques and quality control are crucial to ensure the integrity of the prestressing wires and prevent potential failure due to corrosion or wire breakage. However, all infrastructure has a lifespan and over time the PCCP begins to “breakdown” due to the internal and external pressures, environmental factors, and the substantially continuous flow of water through networked PCCP. Its structural deterioration is complex. Most PCCP failures result from prestressing wire wrap breaks. Failures are often catastrophic with extremely high repair costs. It is estimated each failure of a PCCP segment within a network comes with a price tag of about $2,000,000 to recover.

Currently, PCCP pipes are widely used in national infrastructure projects such as water conservancy, municipal water supply, and water drainage in most large and medium-size cities in the United States. In fact, PCCP is the preferred pipe material for long-distance, large diameter pressurized water pipelines in the world. The maximum diameter of PCCP produced and used is just over seven meters (about 23 feet in diameter), while the internal pressure in the pipe can reach over 2.75 MPa. Networks of PCCP can be buried to a soil cover depth of about 30 meters (almost 100 feet below ground), and the longest current length of a network PCCP is 4500 km (about 2800 miles) in the Libyan “Dagon River” water transmission project in Africa. The total length of PCCP lines in United States is estimated to be about 48,000 km (about 30,000 miles) and in China it is estimated to be 30,000 km (about 18,600 miles) with a ten percent increase annually.

There is an electromagnetic system commercially available to inspect networks of PCCP. The inspection system is based on the principle of remote field eddy current electromagnetic detection. It is widely used in pipelines having small diameters. The electromagnetic system consists of an emitter, which generates electromagnetic field a distance along the pipe axially, and a receiver to catch the electromagnetic energy transmitted through the pipeline. The signal inducted in the receiver represents the pipeline structure condition with given boundary conditions. Furthermore, the signal at the receiver shows distortion when the pipeline is broken or has structural damage. It uses a platform equipped with the electromagnetic system moving in the pipe to conduct inspection in PCCP pipelines.

Pure Technology in Canada first transformed remote field eddy current electromagnetic pipeline detection to P-wave electromagnetic detection in 1997 for inspection of large diameter pipe, such as PCCP pipeline. It was commercialized for broken prestressed steel wire detection in PCCP. However, as shown in FIGS. 2 and 3, the technology depends on well-trained technicians to operate the platform moving in the PCCP with a daily inspection distance limited to a range of from about 1.5 km to 5.6 km (about 1-3.5 miles). This requirement drives the inspection cost very high and results in dewatering any large network of PCCP for too long during the inspection.

Until the invention of the present application, these and other problems in the prior art went either unnoticed or unsolved by those skilled in the art. The system and methods disclosed herein provide an inspection system which performs accurate analysis of the structural “health” of PCCP networks using an associated electronic device and a skillfully built database without sacrificing portability features, design, style or affordability.

### SUMMARY OF THE INVENTION

There is disclosed herein an improved system and method for inspecting networks of PCCP efficiently and effectively. The disclosed system avoids the disadvantages of prior devices while affording additional structural and operating advantages.

Generally speaking, the qualitative testing system for a PCCP pipeline network comprises a database, a computer-controlled pipe robot, and a stored navigational program. The preferred database comprises a plurality of P-wave signal readings associated with structural features of PCCP pipe and facilitates the testing and inspection. The preferred pipe robot is configured with a motor to move autonomously through a PCCP pipeline network and includes P-Wave equipment thereon including a transmitter and a receiver. A computer having memory and programming to control operation of the motor is also fitted to the pipe robot and the navigational system program for the PCCP pipeline network is stored on the memory of the computer. In a preferred embodiment, the pipe robot is moved through the PCCP pipeline network by the computer-controlled motor, guided by the stored navigational system program while the P-wave equipment is continually operating to transmit and receive a signal.

Preferably, in specific embodiments the pipe robot moves at a targeted speed of 5 meters per second. The speed may be optimized, wherein the pipe robot moves at a speed X, wherein X is determined by three variables. Those variables included an offset distance (L) between the emitter and the receiver, a frequency (β) of the transmitter signal (in Hz), and an acquisition frequency (σ) at a measurement point (in Hz).

The relationship between the variables is best characterized by the function:

$$X = f ( \frac{L}{\frac{1.}{β} σ} )$$

In specific embodiments of the disclosed system, an algorithm is provided which:
a. compares the plurality of signals in the memory to P-wave signal readings associated with structural features of PCCP pipe stored in the database;
b. determines any matches between the plurality of signals in the memory and the P-wave signal readings; and
c. relates the associated structural feature of PCCP pipe for each P-wave signal reading to all matching stored signals in the memory.

Regarding the claimed method for building a database to be used for qualitative testing of PCCP structures, a preferred method comprises
(a) setting up a known length of test PCCP having first and second open ends;
(b) positioning a track within the test PCCP extending between the first and second ends;
(c) moving a pipe robot back and forth along the track, the robot comprising P-wave equipment including an emitter for emitting a continuous signal within a PCCP structure and a detector for detecting the continuous signal after being emitted into a PCCP structure;
(d) operating the P-wave equipment while the pipe robot moves back and forth on the track;
(e) detecting a signal by the P-wave equipment detector and simultaneously recording a location of the moving pipe robot within the test PCCP;
(f) storing the detected signal and the recorded location as a plurality of signal readings;
(g) associating each of the plurality of signal readings with a known structural feature of the test PCCP;
(h) storing the plurality of signal readings with the associated known structural features into a database;
(i) manipulating the test PCCP to replicate different structural conditions of PCCP; and
(j) repeating the steps (c) through (i) until all desired structural conditions have been replicated.

The method and system links together technical features, including: 1) conditional data acquisition; 2) a navigable system in which AI algorithms combine inertial navigation (no signal) with GPS PCCP network and mapping; 3) the travel speed of a pipe robot is controlled autonomously according to a position or section of the PCCP pipe; 4) the cumulative error of pipe robot travel in the PCCP pipeline is controlled to not exceed the deviation introduced by traveling in a single PCCP pipe; 5) establishes a model library of the broken wires electromagnetic signal characteristics of the PCCP pipe with directionality (diagnostic direction), including computer simulation and AI algorithm models; 6) ability to calculate signal-to-noise ratio at different PCCP positions between PCCP pipe port and pipe body, and ability to distinguish the difference in real time; 7) establish the functional relationship (controllability) between the emitter and receiver coil offset distance, the frequency of the emitter signal, and the optimal combination of the frequency of the acquisition in the electromagnetic detection equipment, so to improve the optimal (theoretical) diagnostic speed of electromagnetic detection equipment under the condition of not losing any accuracy; 8) improve accuracy owing to prestressing wires in PCCP pipe being directionally spring tightened so that a broken wire has electromagnetic signal characteristics with a directional component which can be used to independently verify from an opposite direction; and 9) build a system cloud platform that includes the PCCP pipeline GPS map, the functional PCCP pipeline in the mapping network, the functional PCCP structure topology diagram and the broken wire model library independent from the pipe type.

These and other aspects of the invention may be understood more readily from the following description and the appended drawings.

### BRIEF DESCRIPTION OF THE DRAWINGS

For the purpose of facilitating an understanding of the subject matter sought to be protected, there are illustrated in the accompanying drawings, embodiments thereof, from an inspection of which, when considered in connection with the following description, the subject matter sought to be protected, its construction and operation, and many of its advantages should be readily understood and appreciated.

FIG. 1 is a perspective view of an embodiment of a Prestressed Concrete Cylinder Pipe (PCCP);

FIG. 2 is an image showing an embodiment of a manual pipe inspection system;

FIG. 3 is another image showing an embodiment of a manual pipe inspection system;

FIG. 4A is an illustration of a cross section of an embodiment of a section of test PCCP in accordance with the disclosed invention;

FIG. 4B is a close-up view of the outer wall of an embodiment of a section of test PCCP showing exposed structural wires;

FIG. 5 is a schematic of an embodiment of a section of test PCCP illustrating signal interference due to structural features of the PCCP;

FIG. 6 is a schematic side view of a test PCCP set-up including a pipe robot with P-wave equipment;

FIG. 7 is a top view of the set-up of FIG. 6;

FIG. 8 is an end view of the set-up of FIG. 6;

FIG. 9 is an illustration of an embodiment of a computer-controlled pipe robot in accordance with the disclosed invention;

FIG. 10 illustrates an exemplary mapping of an existing network of PCCP for which inspection is required; and

FIG. 11 is schematic illustrating an embodiment of the offset feature of the emitter an receiver coils to optimize travel speed of the pipe robot in accordance with the disclosed invention.

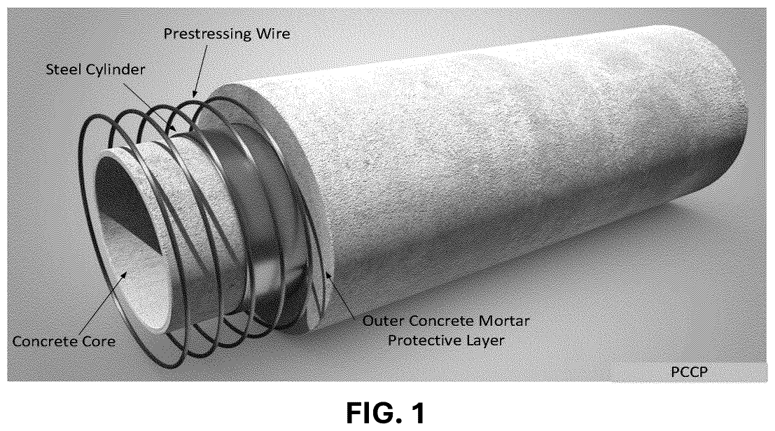

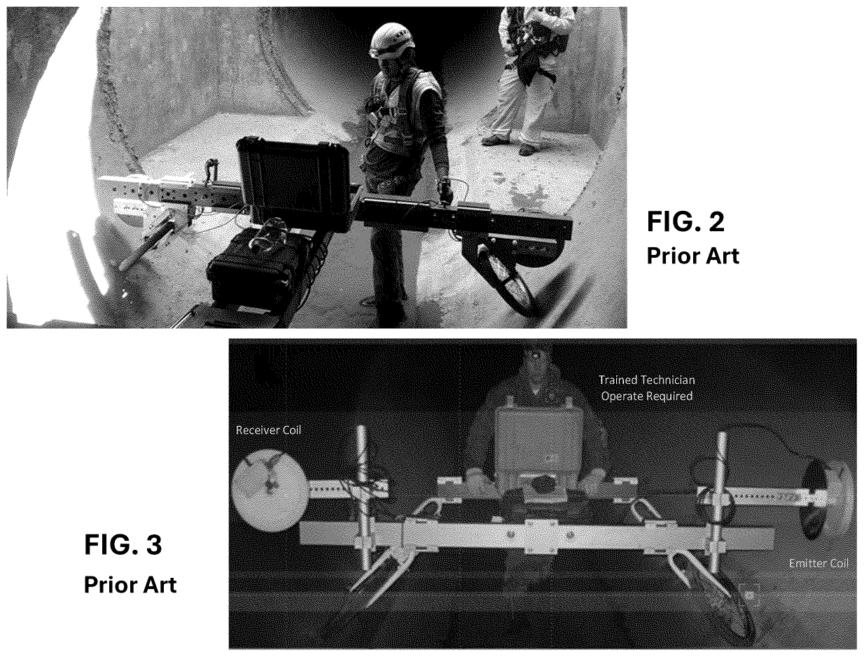

### DETAILED DESCRIPTION OF THE INVENTION

While this invention is susceptible of embodiments in many different forms, there is shown in the drawings and will herein be described in detail at least one preferred embodiment of the invention with the understanding that the present disclosure is to be considered as an exemplification of the principles of the invention and is not intended to limit the broad aspect of the invention to any of the specific embodiments illustrated.

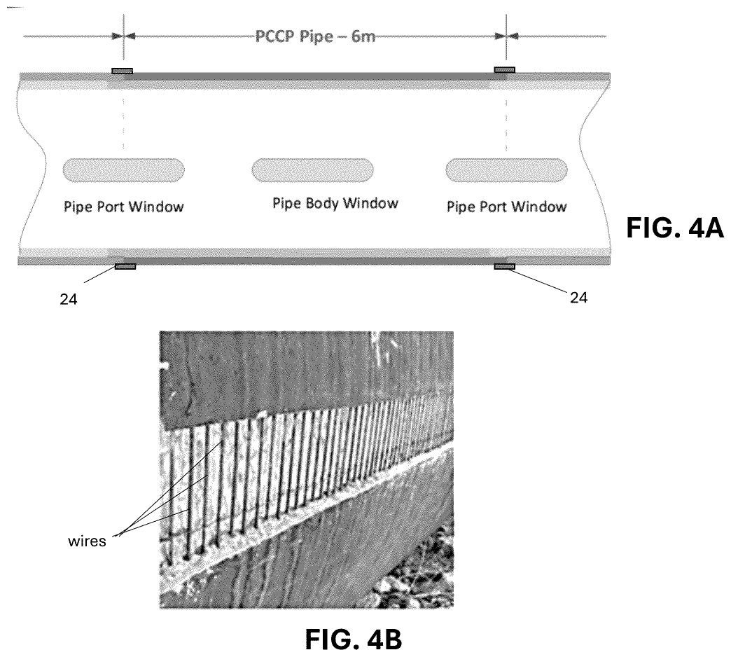

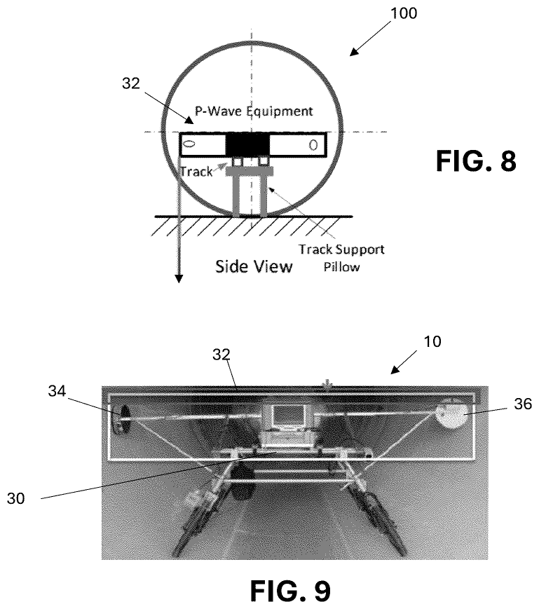

Referring to FIGS. 4-8, a modeling system 100 is illustrated. As will be explained in greater detail below, the modeling system 100 is used to create data to build a substantial database comprising signal data corresponding to known structural features of a PCCP test pipe. The database is then used to facilitate analysis of networks of PCCP.

In FIGS. 9-11, there is at least one embodiment of the disclosed inspection system illustrated, generally designated by the numeral 10. The specifically illustrated system 10 is used for inspection of a network of large diameter PCCP. However, it should be understood that the principles of the invention can be more broadly applied to other types of large-diameter pipes, as long as structural integrity is a paramount issue.

### Building Database Model:

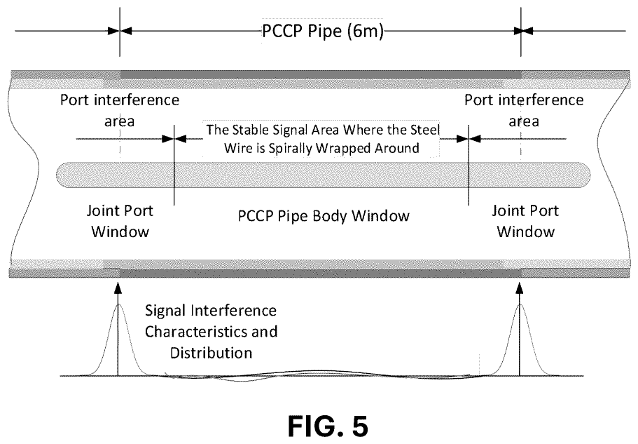

To build a database, the modeling system 100 is employed. The modeling system 100 is comprised of a test PCCP 20 constructed to resemble a standard segment of PCCP. The test PCCP 20 is comprised of at least two and as many as four PCCP sections 22. As each PCCP section 22 is approximately six meters (approx. 19.6 feet) long and anywhere from two to four meters (approx. 6.5 to 13.1 feet) in diameter, two sections 22 are preferred for the test PCCP 20, though more sections 22 may be equally useful. Adjacent sections 22 are typically joined together using pipe collars 24, as shown in FIG. 4A. The collar 24 is one of many structural features which will be recorded during the database building phase of the disclosed invention (see FIG. 5). As the pipeline is thicker at each collar 24, a distinct signal is created and captured at each of these joints. Within the test PCCP 20 is positioned a track 26. The track 26 preferably extends the length of the test PCCP 20 at a height less than half the diameter of the test PCCP 20.

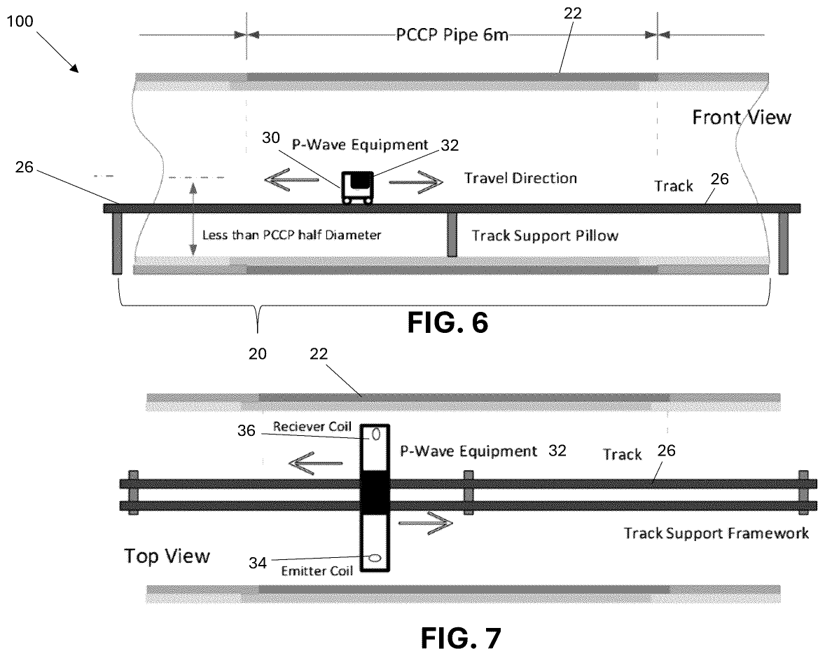

FIGS. 6-8 show an embodiment of the test PCCP 20 and robot set-up for the modeling system 100. To inspect the test PCCP 20, a pipe robot 30 is placed on the track 26 at one end of the test PCCP 20. The pipe robot 30 is comprised of a drive mechanism (not shown) controlled by a computer within the pipe robot 30. For this phase of the invention, the pipe robot 30 may be remote controlled as well. The pipe robot 30 also comprises P-wave equipment 32, including an emitter coil (also referred to as a transmitter or a driver coil) 34 and a receiver coil (also referred to as a detector coil) 36, as illustrated in FIG. 7 (see also, https://www.xylem.com/en-us/brands/pure-technologies/technologies/pipewalker-condition-assessment-platform/).

As is known in the art, the emitter coil 34 is preferably positioned to be perpendicular to the travel direction of the pipe robot 30, while the receiver coil 36 is positioned to be parallel to the direction of travel. This and other possible configurations are disclosed in U.S. Pat. No. 6,781,369 to Paulson et al., the entirety of which is hereby incorporated by reference. A suitable pipe robot may be manufactured and sold by CITIC Heavy Industries Kaicheng Intelligent Equipment Co., Ltd. of China (http://www.citichickc.com/ProductInfoCategory?categoryId=938505).

The pipe robot 30 is then moved—either automatically, manually, or via remote control—back and forth on the track 26 within the test PCCP 20. The P-wave equipment 32 is activated to continuously send and receive an electromagnetic signal during this movement. The signal received is recorded along with the location of the pipe robot 30 within the test PCCP 20 at the time of acquisition.

Referring back to FIGS. 4A and 4B, the test PCCP 20 has initial known structural features, such as material, wall thickness, ports, joints, collars, and the like. These features record differently from their eigenvectors when exposed to the P-wave electromagnetic signal. FIG. 5 illustrates the signal interference characteristic at each of the joints between adjoining PCCP sections. Accordingly, the unique signal, the known structural feature, and the position of each can be readily associated to one another and stored in a database. The repeated back-and-forth movement of the pipe robot 30 and recording of signal data is used to fine tune the signal and position of each known structural feature of the test PCCP 20.

The test PCCP 20 is then manipulated to replicate different structural features and conditions of typical PCCP. These manipulations may include any of the following: cutting one or more structural wires in the test PCCP; altering sidewall density in areas—to mimic construction flaws, for example; altering sidewall thickness—to mimic erosion, for example; cutting holes and/or openings in the sidewall (both internal and external); and/or adding structural anomalies that might include different materials, electronic and/or mechanical devices, equipment, or the like. These manipulations are recorded at specific locations within the test PCCP 20. The back-and-forth movement of the pipe robot 30 on the track 26 and continuous signal emission and reception at the P-wave equipment 32 is repeated in the manipulated test PCCP 20. The signal (amplitude and phase) recordings/readings for the added manipulations are recorded for the known location in the test PCCP 20. This manipulation and recording process can be repeated until all desired structural features and an associated signal are accurately replicated and recorded in the database.

Regarding FIG. 5, a significant benefit of the present system can be better understood. That is, the use of signal-to-noise ratio (SNR) to rapidly distinguish features of the PCCP. As the pipe robot 30 records signals from known structural features of the test PCCP 20, it is able to more quickly identify the signals of those features in subsequent testing based on SNR. This rapid identification of known structural features allows the pipe robot 30 to then move more quickly through a PCCP network, as further explained below. Accordingly, careful attention should be paid to how to use the fundamental equation to simulate other various known structures and material of the PCCP so that the data model database will be independent of such limitations and inspection times can be drastically reduced.

### Inspecting Network of PCCP:

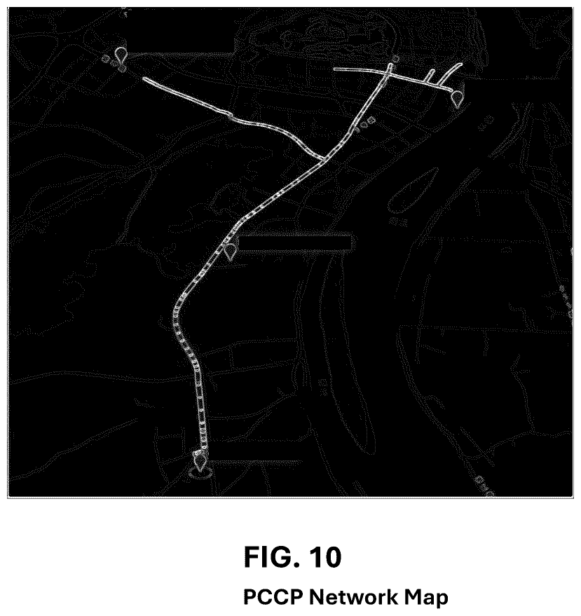

Once the database is completed, it is ready to be used for facilitating inspection of existing PCCP networks. Such networks are comprised of tens to hundreds of miles of pipes, both above and underground. Accordingly, each network of PCCP, an example of which is illustrated in FIG. 10, must be mapped. Construction drawings and data for each PCCP network to be inspected are collected, and each PCCP pipe segment (approx. 6 meters linear) on the drawings is numbered. Using GPS, the numbered PCCP pipe segments are mapped to create a pipeline network model which can be used as a navigational system the first time. This navigational system is then uploaded to a computer-controlled drive system for the pipe robot 30 to create a computer-controlled pipe robot 30. That is, the computer will drive and direction of the pipe robot 30 through a mapped network of PCCP.

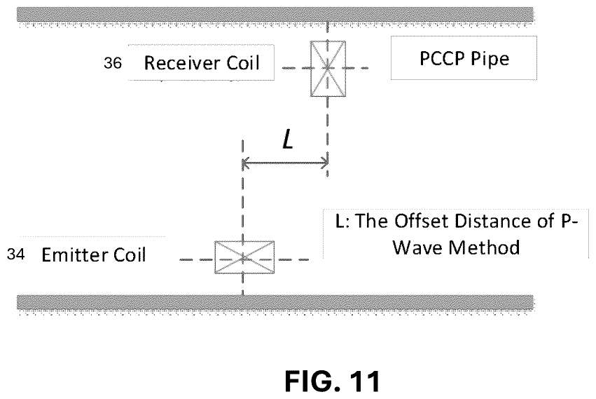

The computer-controlled pipe robot 30 shown in FIG. 9 has an optimal functional relationship between the position of the emitter coil 34 and the receiver coil 36 on the P-wave equipment. The two components are offset a distance (L), as illustrated in FIG. 11. The frequency of the emitter signal (β) and the frequency of the acquisition signal at the equipment (σ), both in Hz, can be used together with the offset distance (L), as three variables, to reference in a look up table (calculate) for a rate of travel (X) of the pipe robot 30. The relationship between X and three variables is set forth below:

$$X = f ( \frac{L}{\frac{1.}{β} σ} ) \qquad ( EQ1 )$$

The exemplary look up table below provides some theoretical data points based on the relation of these variables as set forth in EQ 1 above:

| The offset | Emitter | Acquisition | Travel Speed |
|---|---|---|---|
| distance L (m) | Coil β (Hz) | Point σ (Hz) | X (m/sec) |
| .05 | 25 | 200 | 0.5 |
| .10 | 32 | 100 | 1.5 |
| .15 | 40 | 50 | 2.5 |

With this look up table data, an ideal offset distance (L) could be determined to achieve a desired rate of travel for the robot 30. Ideally, the pipe robot 30 is capable of traveling at a speed of five meters per second, roughly 10 times the current 0.5 meters per second that current manual operators are capable of traveling. Preferably, for some applications the pipe robot 30 may move as fast as about 10 meters per second, with an ability to justify negatively impacting acquisition data quality. That provides an inspection range of at least 18 km per hour (144 km in 8 hours) compared to 6 km per day for manual operators.

From the table above, increasing the offset distance L will reduce the detection resolution for determining a number of broken steel wires; Increasing the frequency of emitter signal β will increase the resistance of signal penetration (travel distance); reducing the frequency of acquisition may result in a loss of information in the acquisition data. These detriments can be justified during the building of the database model (see description above).

For example, the signal-to-noise ratio (SNR) model for PCCP pipe ports (or joints) and pipe body is used to optimize the best combination of emitter and receiver coil offset distances, emitter coil signal frequency, and acquisition frequency from receiver. The port (or joint) of PCCP has a more complex structure than the pipe body, and requires a higher acquisition frequency, a lower frequency of emitter signal, and a shorter offset distance between the emitter coil and receiver. Because the body of each PCCP segment represents about 80% of the PCCP structure, as shown in FIG. 5, broken steel wires will be isolated to these areas. The less complexity of the body portion is therefore much easier and quicker to inspect for the pipe robot. However, being able to know where the other 20% is located—i.e., the pipe ports—is the key to more efficient inspection. The SNR provides that knowledge due to the spike in interference recorded at the pipe port sections (see FIG. 5). The inspection system 10 ability to quickly identify the position of pipe ports (and other known structural features with high interference/noise recordings), allows the pipe robot 30 to be operated in a much more efficient way, thus speeding inspection and lowering inspection time.

As data points/readings are being acquired by the P-wave equipment, AI can be used to apply an algorithm which compares the plurality of signals in the computer memory to P-wave signal readings associated with structural features of PCCP pipe stored in the database. The algorithm determines any matches between the plurality of signals in the memory (i.e., those from the test PCCP) and the P-wave signal readings and relates the associated structural feature of PCCP pipe for each P-wave signal reading to all matching stored signals in the memory. Once a match is found, the structural feature associated with that reading in the database is attached to the location of the acquisition signal from the network pipe inspection based on the navigational map (see above).

As previously noted, the structural feature associated with the specific location of the network pipe inspected could indicate a change in PCCP density, a broken wire, internal wear, or other structural regularities and anomalies. Additionally, the data gathered during inspection can be further used for growing and refinement of the database.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. A method for building a database for qualitative testing of PCCP structures, the method comprising:
a. setting up a known length of test PCCP having first and second open ends;
b. positioning a track within the test PCCP extending between the first and second ends;
c. moving a pipe robot back and forth along the track, the robot comprising P-wave equipment including an emitter coil for emitting a continuous signal within a PCCP structure and a detector (receiver coil) for detecting the continuous signal after being emitted into a PCCP structure;
d. operating the P-wave equipment while the pipe robot moves back and forth on the track;
e. detecting a signal by the P-wave equipment detector and simultaneously recording a location of the moving pipe robot within the test PCCP;
f. storing the detected signal and the recorded location as a plurality of signal readings;
g. associating each of the plurality of signal readings with a known structural feature of the test PCCP;
h. storing the plurality of signal readings with the associated known structural features into a database;
i. manipulating the test PCCP to replicate different structural conditions of PCCP; and
j. repeating the steps (c) through (i) until all desired structural conditions have been replicated.

2. The method of claim 1, wherein manipulating the test PCCP comprises at least one of the following:
cutting at least one structural wire in the test PCCP;
altering sidewall density of the test PCCP;
altering sidewall thickness of the test PCCP;
creating openings in sidewall of the test PCCP; and
adding structural anomalies to the test PCCP.

3. The method of claim 2, wherein the pipe robot moves using a computer-controlled motor.

4. The method of claim 3, wherein the pipe robot moves at a speed of up-to 5 meters per second.

5. The method of claim 1, further comprising using the database to determine structural features of a network of PCCP.

6. The method of claim 5, wherein the step of using the database to provide information on structural features of a network of PCCP comprises:
positioning the pipe robot within the network of PCCP;
moving the pipe robot through the network of PCCP;
operating the P-wave equipment while the pipe robot moves within the network of PCCP;
detecting a signal by the P-wave equipment detector and simultaneously recording a location of the moving pipe robot within the network of PCCP;
storing the detected signal and the recorded location at a plurality of locations within the network of PCCP as a plurality of new signal readings;
comparing each of the plurality of new signal readings to the plurality of stored signal readings in the database to find a matching stored signal reading for each of the plurality of new signal readings; and
associating the known structural feature of a stored signal reading to each matching new signal reading to create an analysis of the network of PCCP.

7. The method of claim 6, further comprising mapping the known structural feature to the location within the network of PCCP of each matching new signal reading.

8. The method of claim 3, further comprising using the database to determine structural features of a network of PCCP.

9. The method of claim 8, wherein the step of using the database to provide information on structural features of a network of PCCP comprises:
positioning the pipe robot within the network of PCCP;
moving the pipe robot through the network of PCCP;
operating the P-wave equipment while the pipe robot moves within the network of PCCP;
detecting a signal by the P-wave equipment detector and simultaneously recording a location of the moving pipe robot within the network of PCCP;
storing the detected signal and the recorded location at a plurality of locations within the network of PCCP as a plurality of new signal readings;
comparing each of the plurality of new signal readings to the plurality of stored signal readings in the database to find a matching stored signal reading for each of the plurality of new signal readings; and
associating the known structural feature of a stored signal reading to each matching new signal reading to create an analysis of the network of PCCP.

10. The method of claim 9, further comprising mapping the known structural feature to the location within the network of PCCP of each matching new signal reading.

11. The method of claim 1, further comprising determining a signal-to-noise ratio at the detector of the P-wave equipment.

12. The method of claim 6, further comprising determining a signal-to-noise ratio at the detector of the P-wave equipment.

13. The method of claim 11, further comprising using the signal-to-noise ratio to distinguish between areas of the test PCCP.

14. The method of claim 13, wherein the areas of the test PCCP are comprised of at least body sections and port sections.

15. The method of claim 12, wherein the signal-to-noise ratio determination allows the pipe robot to move more quickly through areas of the network of PCCP.

16. A qualitative testing system for a PCCP pipeline network, the system comprising:
a database comprising a plurality of P-wave signal readings associated with structural features of PCCP pipe;
a pipe robot configured with a motor to move autonomously through a PCCP pipeline network and having P-Wave equipment thereon including a transmitter and a receiver;
a computer having memory and programming to control operation of the motor; and
a navigational system program for the PCCP pipeline network stored on the memory of the computer;
wherein the pipe robot is moved through the PCCP pipeline network by the computer-controlled motor, guided by the stored navigational system program while the P-wave equipment is continually operating to transmit and receive a signal.

17. The qualitative testing system of claim 16, wherein the pipe robot moves at a speed of 5 meters per second.

18. The qualitative testing system of claim 16, wherein the pipe robot moves at a speed X, wherein X is determined by a relation defined as:
$$X = f ( \frac{L}{\frac{1.}{β} σ} ) ,$$
wherein L is an offset distance between the emitter and receiver; β is a frequency of the transmitter signal (in Hz) and σ is an acquisition frequency (in Hz) at a measurement point.

19. The system of claim 17, wherein the P-wave equipment receiver receives a plurality of P-wave signals during operation and stores the plurality of signals in the memory of the computer-controlled motor.

20. The system of claim 19, further comprising an algorithm which:
a. compares the plurality of signals in the memory to P-wave signal readings associated with structural features of PCCP pipe stored in the database;
b. determines any matches between the plurality of signals in the memory and the P-wave signal readings; and
c. relates the associated structural feature of PCCP pipe for each P-wave signal reading to all matching stored signals in the memory.
